(19) United States
(12) Patent Application Publication
(10) Pub. No.: US 2004/0167438 A1
(43) Pub. Date: Aug. 26, 2004

(54) Reinforced medical device

(76) Inventor: James S. Sharrow, Bloomington, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/375,494

(22) Filed: Feb. 26, 2003

(51) Int. Cl. A61B 5/00

(52) U.S. Cl. 600/585

(57) ABSTRACT

A medical device and methods of making and using the same. The invention may include a shaft having a proximal region and a distal region. A jacket may be disposed on the proximal region. A reinforcing member or braid may be disposed over at least a portion of the jacket.

### FIELD OF THE INVENTION

The invention pertains to medical devices and, more particularly, to medical devices, such as guidewires or the like, having improved torquability characteristics.

### BACKGROUND

A wide variety of medical devices have been developed for medical use, for example, intravascular use. Some of these devices include guidewires, or the like, that have certain torquability characteristics. Of the known medical devices that have defined torquability characteristics, each has certain advantages and disadvantages. There is an ongoing need to provide alternative designs and methods of making and using medical devices with desirable torquability characteristics.

### BRIEF SUMMARY

The invention provides design, material, and manufacturing method alternatives for medical devices having certain torquability characteristics. In at least some embodiments, the medical devices include an elongate shaft or core member that has a proximal portion and a distal portion. The proximal portion may include a coating and a reinforcing member such as a braid or coil may be disposed on or within at least a portion of the coating. Some of the other features and characteristics of example medical devices are described in more detail below.

### BRIEF DESCRIPTION OF THE DRAWINGS

FIG. 1 is a partially cut-away perspective view of an example medical device;

FIG. 2 is a cross-sectional view of the example medical device of FIG. 1;

FIG. 3 is a cross-sectional view of an alternative example medical device;

FIG. 4 is a cross-sectional view of an alternative example medical device;

FIG. 5 is a cross-sectional view of an alternative example medical device; and

FIG. 6 is a cross-sectional view of an alternative example medical device.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

FIG. 1 is a partially cut-away perspective view of an example medical device 10. In at least some embodiments, device 10 may be a guidewire that, for example, includes a reinforcement member or braid 12 disposed adjacent its proximal region. Braid 12 may provide device 10 with a number of desirable features. For example, braid 12 may help transmit torque or otherwise convey forces from the proximal end of device 10 toward the distal end. Some of the features and characteristics of device 10 as well as braid 12 are described in more detail below. It should be noted that although the embodiments shown in FIG. 1 and other figures show device 10 as a guidewire, this is not intended to be limiting. Device 10 could be essentially any medical device or be any device designed to pass through an opening or body lumen that includes a solid shaft or includes a solid core portion and/or core member. For example, device 10 may comprise a core wire (for use alone or with a variety of other medical devices), catheter (e.g., therapeutic or diagnostic catheters), endoscopic device, laproscopic device, an embolic protection device, and the like, components of any of these devices, or any other suitable device including a solid core portion. In such devices, a reinforcing member can be disposed on or within a coating that is disposed over a portion of the solid shaft or core wire, for example, to enhance the tortional response of such devices.

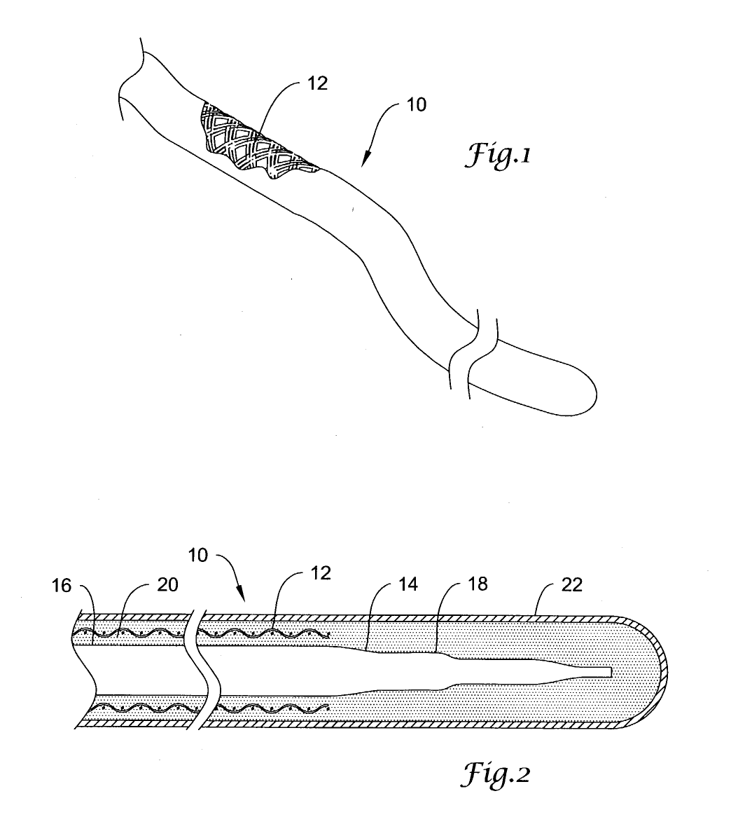

A partial cross-sectional view of device 10 is shown in FIG. 2. Here it can be seen that device 10 may include a core wire or member 14 having a proximal region 16 and a distal region 18. Core wire 14 can be made of any suitable materials including metals, metal alloys, polymers, elastomers, or the like, or combinations or mixtures thereof. Some examples of suitable metals and metal alloys include stainless steel, such as 304v stainless steel; nickel-titanium alloy, such as linear elastic or superelastic (i.e., pseudoelastic) nitinol, nickel-chromium alloy, nickel-chromium-iron alloy, cobalt alloy, tungsten, tungsten alloy, Elgiloy, MP35N, or the like; or other suitable material. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

Within the family of commercially available nitinol alloys, is a category designated “linear elastic” which, although is similar in chemistry to conventional shape memory and superelastic varieties, exhibits distinct and useful mechanical properties. By skilled applications of cold work, directional stress, and heat treatment, the wire is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range.

For example, in some embodiments, there is no martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “pushability” around tortuous anatomy.

In some embodiments, the linear elastic nickel-titanium alloy is in the range of about 50 to about 60 weight percent nickel, with the remainder being essentially titanium. In some particular embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. In some other embodiments, a superelastic alloy, for example a superelastic nitinol can be used to achieve desired properties.

In at least some embodiments, portions or all of core wire 14 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like.

In some embodiments, a degree of MRI compatibility is imparted into device 10. For example, to enhance compatibility with Magnetic Resonance Imaging (MRI) machines, it may be desirable to make core wire 14, or other portions of the medical device 10, in a manner that would impart a degree of MRI compatibility. For example, core wire 14, or portions thereof, may be made of a material that does not substantially distort the image and create substantial artifacts (artifacts are gaps in the image). Certain ferromagnetic materials, for example, may not be suitable because they may create artifacts in an MRI image. Core wire 14, or portions thereof, may also be made from a material that the MRI machine can image. Some materials that exhibit these characteristics include, for example, tungsten, Elgiloy, MP35N, nitinol, and the like, and others.

The entire core wire 14 can be made of the same material, or in some embodiments, can include portions or sections made of different materials. In some embodiments, the material used to construct core wire 14 is chosen to impart varying flexibility and stiffness characteristics to different portions of core wire 14. For example, proximal region 16 and distal region 18 may be formed of different materials, for example materials having different moduli of elasticity, resulting in a difference in flexibility. In some embodiments, the material used to construct proximal region 16 can be relatively stiff for pushability and torqueability, and the material used to construct distal region 18 can be relatively flexible by comparison for better lateral trackability and steerability. For example, proximal region 16 can be formed of straightened 304v stainless steel wire or ribbon, and distal region 18 can be formed of a straightened super elastic or linear elastic alloy, for example a nickel-titanium alloy wire or ribbon.

In embodiments where different portions of core wire 14 are made of different material, the different portions can be connected using any suitable connecting techniques. For example, the different portions of the core wire can be connected using welding (including laser welding), soldering, brazing, adhesive, or the like, or combinations thereof. Additionally, some embodiments can include one or more mechanical connectors or connector assemblies to connect the different portions of the core wire that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276 filed on Oct. 5, 2001 and Ser. No. 10/068,992 filed on Feb. 28, 2002, which are incorporated herein by reference. Some additional examples of suitable interconnection techniques are disclosed in a U.S. patent application entitled “COMPOSITE MEDICAL DEVICE” (Attorney docket number 1001.1546101) filed on even date herewith, which is also incorporated herein by reference.

The length of core member 14 (and/or device 10), or the length of individual portions thereof, are typically dictated by the length and flexibility characteristics desired in the final medical device. For example, proximal region 16 may have a length in the range of about 20 to about 300 centimeters or more and distal region 18 may have a length in the range of about 3 to about 50 centimeters or more. It can be appreciated that alterations in the length of portions 16/18 can be made without departing from the spirit of the invention.

Core wire 14 can have a solid cross-section, but in some embodiments, can have a hollow cross-section. In yet other embodiments, core wire 14 can include a combination of areas having solid cross-sections and hollow cross sections. Moreover, core 14, or portions thereof, can be made of rounded wire, flattened ribbon, or other such structures having various cross-sectional geometries. The cross-sectional geometries along the length of shaft 14 can also be constant or can vary. For example, FIG. 2 depicts core wire 14 as having a round cross-sectional shape. It can be appreciated that other cross-sectional shapes or combinations of shapes may be utilized without departing from the spirit of the invention. For example, the cross-sectional shape of core wire 14 may be oval, rectangular, square, polygonal, and the like, or any suitable shape.

As shown in FIG. 2, distal region 18 may include one or more tapers or tapered regions. In some embodiments distal region 18 may be tapered and have an initial outside size or diameter that can be substantially the same as the outside diameter of proximal region 16, which then tapers to a reduced size or diameter. For example, in some embodiments, distal region 18 can have an initial outside diameter that is in the range of about 0.010 to about 0.040 inches, that tapers to a diameter in the range of about 0.001 to about 0.005 inches. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. Although FIG. 2 depicts distal region 18 of core wire 14 as being tapered, it can be appreciated that essentially any portion of core wire 14 may be tapered and the taper can be in either the proximal or the distal direction. As shown in FIG. 2, the tapered region may include one or more portions where the outside diameter is narrowing, for example, the tapered portions, and portions where the outside diameter remains essentially constant, for example, constant diameter portions. The number, arrangement, size, and length of the narrowing and constant diameter portions can be varied to achieve the desired characteristics, such as flexibility and torque transmission characteristics. The narrowing and constant diameter portions as shown in FIG. 2 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, core wire 14 can be centerless ground using a Royal Master HI-AC centerless grinder. Some examples of suitable grinding methods are disclosed in U.S. patent application Ser. No. 10/346,698 filed Jan. 17, 2003, which is herein incorporated by reference.

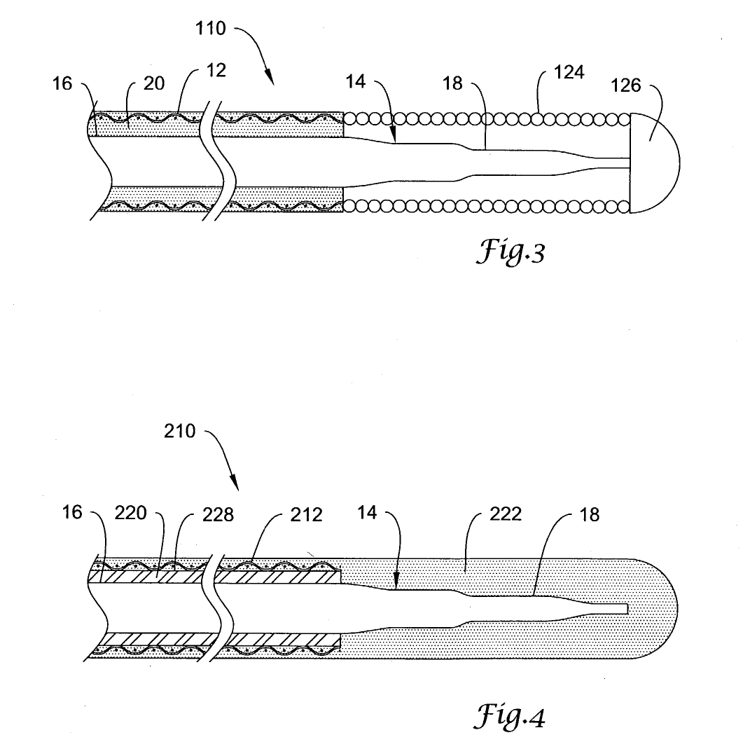

FIG. 2 also illustrates that a jacket 20 may be disposed over core wire 14. In some embodiments, jacket 20 is disposed over essentially the entire length of core wire 14 and may, for example, extend distally beyond distal region 18. Alternatively, jacket 20 may be disposed over only portions of core wire (e.g., over only proximal region 16 similar to what is shown in FIG. 3). For example, jacket 20 may be disposed over the proximal 9/10, ⅘, ¾, ⅔, ½, or ¼ of the length of core wire 14. In some embodiments, jacket 20 may extend to the very proximal end of the core, while in other embodiments, the jacket 20 may end at a point distal of the proximal end of the core.

Jacket 20 may be made of any suitable material including those listed herein. For example, jacket 20 may be polymeric or otherwise include a polymer. Polymers may include high performance polymers having the desired characteristics such as flexibility and torquability. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysufone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELL®), polyolefin, polyetheretherketone (PEEK), polyimide (PI), polyetherimide (PEI), nylon, other suitable materials, or mixtures, combinations, copolymers thereof, polymer/metal composites, lubricous polymers, and the like. In some embodiments jacket 20 can include a liquid crystal polymer (LCP) blended with other polymers to enhance torqueability. For example, the mixture can contain up to about 5% LCP. This has been found to enhance torqueability.

Jacket 20 may be formed, for example, by coating, by extrusion, co-extrusion, interrupted layer co-extrusion (ILC), fusing or bonding one or more preformed polymer segments to core member 14, or any other appropriate method. The layer may have a uniform stiffness or a gradual reduction in stiffness from the proximal end to the distal end thereof. The gradual reduction in stiffness may be continuous as by ILC or may be stepped as by fusing together separate extruded tubular segments. Jacket 20 may be impregnated with a radiopaque filler material to facilitate radiographic visualization. Those skilled in the art will recognize that these materials can vary widely without deviating from the scope of the present invention.

Reinforcement member or braid 12 may comprise a braid of interwoven strands. Braid can be of any appropriate size and shape for use in the particular medical device into which it will be incorporated. As shown in FIG. 2, braid 12 may have a generally circular cross-sectional shape, and is appropriately sized for use in an intravascular guidewire. A broad variety of other shapes and sizes could be used, depending upon the intended use and desired characteristics of braid 12. For example, in some embodiments, braid 12 could have a flat, curved, oval, or multisided cross-sectional shape, for example, triangular, square, rectangular, pentagonal, hexagonal, and so fourth.

Furthermore, braid 12 can be formed using any suitable technique for forming the appropriate reinforcing structure. Braid 12 can be formed using a suitable number of strands or filaments. The number of strands or filaments used may often depend upon the desired characteristics of braid 12, and the patterns or techniques used to form braid 12. In some embodiments, between one and thirty-two, or even more, strands may be used in each direction.

In some embodiments, the braid reinforcement member 12 can include an equal number of strands wound in each direction at the same pitch. In other words, the same number of strands are wound in opposite directions at the same pitch. Some other embodiments may include a braid reinforcement layer with an unequal number of strands wound in each direction. The strands in each direction may be wound at the same pitch or at differing pitches. Some examples of structures of reinforcing members 12 can be found in U.S. patent application Ser. No. 10/346,697, filed on Jan. 17, 2003 entitled “Unbalanced Reinforcing Members for Medical Device”, which is incorporated herein by reference. The braid density may also vary widely; in some embodiments, the braid density may be as low as about 10 pic; while in other embodiments braid density may increase to the range of about 300 pic.

The strands or filaments that collectively define braid 12 may be appropriately sized and shaped depending upon the desired characteristics of braid 12 and pattern used. In some embodiments, the cross-sectional shape of the filaments can be circular, oval, flat, or multisided, for example, triangular, square, rectangular, pentagonal, hexagonal, and so fourth. In other embodiments, the filaments may be formed as ribbons.

In some other embodiments, reinforcing member 12 can be a non-braided structure, for example a coil, that is disposed over, or embedded within jacket 20, as discussed below. The coil may be made of a variety of materials including metals, metal alloys, polymers, and the like, as discussed in reference to the core wire. Some examples of material for use in the coil include materials such as those used for a braid, for example, high performance polymers, stainless steel, nickel-chromium alloy, nickelchromium-iron alloy, cobalt alloy, tungsten, tungsten alloy, Elgiloy, MP35N, or the like, or other suitable materials. Some additional examples of suitable material include straightened super elastic (i.e., pseudoelastic) or linear elastic alloy (e.g., nickel-titanium) material, or alternatively, a polymer material, such as a high performance polymer. In some embodiments, the coil can be made or include a radiopaque materials, as discussed herein, such as gold, platinum, tungsten, or the like, or alloys thereof. The coil may be formed of round or flat ribbon or other geometries ranging in dimensions to achieve the desired flexibility. In some embodiments, the coil may be a round wire in the range of about 0.001-0.015 inches in diameter. The coil may be wrapped in a helical fashion by conventional winding techniques. The pitch of adjacent turns of coil may be tightly wrapped so that each turn touches the succeeding turn or the pitch may be set such that coil is wrapped in an open fashion.

Reinforcing member or braid 12 may be disposed over at least a portion of jacket 20. In at least some embodiments, braid 12 may be partially or fully embedded within jacket 20. Embedding may be accomplished in a number of ways. For example, braid 12 may be placed over a partially molten jacket 20 and then placing additional partially molten jacket 20 over braid 12. Alternatively, braid 12 can be disposed over jacket 20, additional jacket layer or layers can be placed over braid 12, and then the various layers of jacket 20 can be melted together. In still other alternatives, jacket 20 may comprise a plurality of heat shrinkable materials such that braid 12 may be disposed between two or more layers of jacket 20 and then the jacket layers 20 can be shrunk and melted together. In still other alternative embodiments, jacket 20 may include a low melting temperature polymer that flows when exposed to heat. Braid 12 can be disposed over jacket 20 and a heat shrink outer jacket or coating can be disposed over braid 12 and the various structures can be thermally treated to embed braid in jacket 20. The outer coating can be left on the outer surface or it may be subsequently removed. It can be appreciated that a number of other manufacturing methods may be used to embed braid 12 within jacket 20 (and/or layers of jacket 20) without departing from the spirit of the invention.

It can be appreciated that by disposing the reinforcing member, for example braid 12 or a coil, on or embedding it within jacket 20 can result in braid 12 being radially spaced away from core wire 14. This feature may be desirable, for example, because it is believed that spacing braid 12 away from the centerline of core wire 14 improves the torque transmission from proximal region 16 toward distal region 18. There may also be a number of additional desirable features or characteristics associated with spacing braid 12 from core wire 14.

In addition to or as an alternative to being spaced from core wire 14, braid 12 may also improve torque transmission based on its material composition and configuration. For example, braid 12 may be comprised of a strong or high modulus material such as aramid (also known as poly-para-phenylene terephthalamide such as, for example, KEVLAR®, which is commercially available from DuPont). Alternatively, braid 12 or the filaments making up the braid may be made of other materials such as polymers, metals, metal alloys, or combinations thereof, for example like those materials disclosed above with reference to materials useable for the core wire 14. For example, braid 12 may include a first filament made from a combination of materials, or braid 12 may include a first filament made of a first material and a second filament made from a second material. In some embodiments, the material of braid 12 can be blended with a liquid crystal polymer (LCP). For example, the mixture can contain up to about 5% LCP. This has been found to enhance torqueability. In some other embodiments, braid 12 can include combinations of filaments or strands made up of different types of materials. Also, braid 12 can include radiopaque materials or materials that are MRI compatible as discussed above.

In some embodiments, the reinforcing member, for example braid 12 or a coil, extends over the entire length of jacket 20, while in other embodiments, braid 12 extends over only a portion of jacket 20. In some embodiments, braid 12 extends only about jacket 20 disposed on the proximal region 16 or core wire 14. For example, braid 12 may be disposed about the proximal 9/10, ⅘, ¾, ⅔, ½, or ¼, of core wire 14. In some embodiments, braid 12 may extend to the very proximal end of core wire 14, while in other embodiments, the proximal end of braid 12 may end at a point distal of the very proximal end of core wire 14. As such, the length of reinforcing member 12 can vary. In some embodiments, reinforcing member 12 has a length in the range of about 50 to about 450 centimeters or longer. It should be understood that these lengths can vary, depending upon, for example, the length of the particular device and upon the desired characteristics.

In some embodiments, a second or outer jacket 22, for example a lubricious, a hydrophilic, a protective, or other type of coating may be applied over portions or all of device 10. Some examples of materials that can be used for the outer coatings include those discussed above with reference to jacket 20. The inner and outer jackets or coatings can be the same or different. Some embodiments can include more than one inner or outer jackets. In some embodiments, the outermost jacket can be a lubricious or hydrophilic coating. Additionally, different coatings can be applied to different sections of device 10. Hydrophobic coatings such as fluoropolymers provide a dry lubricity which can improve guidewire handling and device exchanges. Lubricious coatings can also improve steerability and lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Another example medical device 110 is shown in FIG. 3. Device 110 is similar to device 10, except that it includes a spring tip characterized by a distal coil 124 and solder ball tip 126. In at least some embodiments, coil 124 may be disposed over distal region 18. However, coil 124 may be disposed at essentially any appropriate position and have essentially any appropriate length. Coil 124 may be made of essentially any material including any of those listed herein. For example, coil 124 may be generally metallic and may include a radiopaque material. Variations in the material composition, cross-sectional shape, length, pitch, and other characteristics are within the scope of the invention. It should also be understood that other structures, such as marker bands or coils, shaping or safety ribbons or wires, or additional coils or polymer layers can be included in this and other embodiments.

FIG. 4 is a partial cross-sectional view of device 210. Device 210 is similar to other devices described herein, except that braid 212 is disposed on an exterior surface 228 of jacket 220. According to this embodiment, device 210 may be manufactured by disposing jacket 220 on core wire 14, for example adjacent proximal region 16. Braid 212 may be disposed on exterior surface 228 of jacket 220. In some embodiments, braid 212 may be attached to jacket 220 by a suitable connecting means such as adhesive, any of the various thermal bonding, mechanical or frictional bonding, and the like.

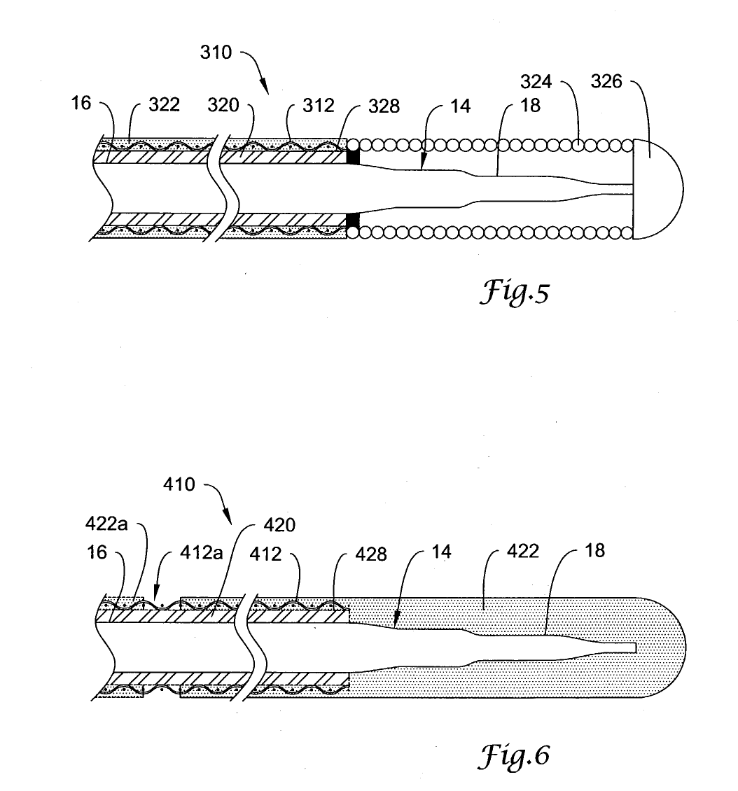

Alternatively or in addition to what is described above, braid 212 may be attached or otherwise coupled to jacket 220 by second jacket or coating 222. Second coating 222, which may be similar to second coating 22, may further extend distally of jacket 220 and, for example, extend over distal region 18 of core wire 14. Accordingly, jacket 220 may form or otherwise define an atraumatic distal tip to device 210. Of course, other forms of tips may be substituted. For example, FIG. 5 is a partial cross-sectional view of device 310 that is similar to device 210 except that includes a spring tip.

Similar to what is described above, the spring tip of device 310 may include coil 324 and solder ball 326. Coil 324 may vary similar to what is described above in relation to coil 224. Device 310 also may include braid 312 disposed on exterior surface 328 of jacket 320. In some embodiments second jacket or coating 322 may be disposed over braid 312.

FIG. 6 is a partial cross-section view of device 410. Device 410 is similar to other devices described herein. For example, device 410 may include braid 412 being disposed on exterior surface 428 of jacket 420 and second jacket or coating 422 being disposed over braid. However, a portion of second coating 422 may be absent along an exposed portion of braid 12, as indicated by reference number 412a. This exposed braid portion 412a may define a portion of the exterior surface of device 410 and may be desirable for a number of reasons. For example, exposed braid portion 412a may have a generally rough or consistent texture that may enhance the ability of a clinician to effectively grasp and actuate device 410.

Exposed braid portion 412a may be defined or “exposed” in a number of ways. For example, exposed braid portion 412a may be defined by truncating a portion of coating 422 so that portion 412a is left uncoated. Alternatively, a portion of second coating 422 may be removed (e.g., mechanically, chemically, thermally, and the like, or any other appropriate way) so as to expose portion 412a. In some embodiments, a proximal section 422a (indicated in phantom line) of second coating 422 may be disposed over braid 412. Proximal section 422a may be defined upon removing a portion of second coating 422 to expose braid portion 412a. Alternatively, proximal section 422a may be disposed proximally of portion 412a so as to define a structural element that is distinct from second coating 422.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A guidewire, comprising:
an elongate core member having a proximal region and a tapered distal region;
a jacket disposed on at least the proximal region of the core member; and
a braid disposed over at least a portion of the jacket.

2. The guidewire of claim 1, wherein core member includes nickel-titanium alloy.

3. The guidewire of claim 1, wherein the jacket includes a polymer.

4. The guidewire of claim 1, wherein the proximal region includes a proximal end and a distal end, and wherein the jacket extends from a proximal end of the proximal region to the distal end of the proximal region.

5. The guidewire of claim 1, wherein the braid includes a metal.

6. The guidewire of claim 1, wherein the braid includes a non-metallic material.

7. The guidewire of claim 1, wherein the braid includes a polymeric material.

8. The guidewire of claim 1, wherein the braid includes poly-para-phenylene terephthalamide.

9. The guidewire of claim 1, wherein at least a portion of the braid is embedded within the jacket.

10. The guidewire of claim 9, further comprising a second coating disposed over the jacket.

11. The guidewire of claim 10, wherein the second coating comprises a layer of polymer material.

12. The guidewire of claim 1, wherein the braid is disposed on an outside surface of the jacket.

13. The guidewire of claim 12, wherein at least a portion of the braid defines an outer surface of the guidewire.

14. The guidewire of claim 12, further comprising a second coating disposed over the braid.

15. The guidewire of claim 14, wherein the second coating comprises a layer of polymer material.

16. A medical device, comprising:
an elongate shaft including a solid core member;
the core member including a proximal region and a distal region;
a polymeric jacket disposed on the core member; and
a braid disposed over at least a portion of the jacket.

17. The medical device of claim 16, wherein core member includes nickel-titanium alloy.

18. The medical device of claim 16, wherein the braid includes a metal.

19. The medical device of claim 16, wherein the braid includes a polymeric material.

20. The medical device of claim 16, wherein the braid includes poly-paraphenylene terephthalamide.

21. The medical device of claim 16, wherein at least a portion of the braid is embedded within the jacket.

22. The medical device of claim 21, further comprising a second coating disposed over the jacket.

23. The medical device of claim 16, wherein the braid is disposed on an outside surface of the jacket.

24. The medical device of claim 23, wherein at least a portion of the braid defines an outer surface of the medical device.

25. The medical device of claim 23, further comprising a second coating disposed over the braid.

26. A method for manufacturing a guidewire, comprising the steps of:
providing an elongate core member having a proximal region and a tapered distal region;
disposing a polymeric jacket on the proximal region; and
disposing a braid over at least a portion of the jacket.

27. The method of claim 26, wherein the step of disposing a braid over at least a portion of the jacket includes embedding the braid in the jacket.

28. The method of claim 27, further comprising the step of disposing a second coating over the jacket.

29. The method of claim 26, wherein the step of disposing a braid over at least a portion of the jacket includes disposing the braid on an outside surface of the jacket.

30. The method of claim 29, further comprising the step of disposing a second coating over the braid.

31. The method of claim 29, wherein the step of disposing the braid on an outside surface of the jacket includes defining an exterior surface of the guidewire with the braid.

32. A guidewire, comprising:
a solid core member having a proximal section and a distal section;
a polymeric jacket disposed over the proximal section; and
means for transmitting torque from the proximal section to the distal section, the means for transmitting torque being disposed over at least a portion of the jacket.

33. A guidewire, comprising:
a solid core member having a proximal section and a distal section;
a polymeric jacket disposed over the proximal section; and
means for conveying force from the proximal section to the distal section, the means for transmitting torque being disposed over at least a portion of the jacket.

34. A guidewire, comprising:
an elongate shaft including a solid core member;
the core member including a proximal region and a distal region;
a polymeric jacket disposed on the core member;
a reinforcing member disposed over at least a portion of the jacket; and
wherein the reinforcing member comprises a coil or braid.

35. The guidewire of claim 34, wherein the reinforcing member comprises a coil.

36. The guidewire of claim 34, wherein the shaft includes stainless steel, nickel-titanium alloy, nickel-chromium alloy, nickel-chromium-iron alloy, cobalt alloy, tungsten, tungsten alloy, Elgiloy, MP35N, a high performance polymer, or combinations thereof.
